(19) United States
(12) Patent Application Publication
(10) Pub. No.: US 2002/0192231 A1
(43) Pub. Date: Dec. 19, 2002

(54) Method of increasing anti-neuGc antibody levels in blood

(75) Inventors: Alex Zhu, New York, NY (US); Shiming Zhang, Danville, PA (US)

Correspondence Address: COHEN, PONTANI, LIEBERMAN & PAVANE, Suite 1210, 551 Fifth Avenue, New York, NY, 10176, US

(73) Assignee: Immucom Inc.

(21) Appl. No.: 10/154,046

(22) Filed: May 23, 2002

Related U.S. Application Data
(60) Provisional application No. 60/293,244, filed on May 24, 2001.
(60) Provisional application No. 60/297,692, filed on Jun. 12, 2001.

(51) Int. Cl. A61K 39/00; A61K 31/739

(52) U.S. Cl. 424/185.1; 424/277.1; 514/54

(57) ABSTRACT

The present invention provides for a method of increasing anti-NeuGc antibody level in the blood of an animal by either intravenous injection of autogenic or allogeneic cells, wherein the cells contain NeuGc on their plasma membrane, or oral administration of NeuGc-containing substance from animal tissues. This method may be applied to the immunoprevention of humans who have a higher risk of developing cancer or the prevention of recurrence in cancer patients who have had therapeutic treatment. This method may further be applied to the immunoprevention of chickens at risk of developing Marek's Disease.

### RELATED APPLICATIONS

This application claims priority from U.S. Provisional Patent Applications Serial Nos. 60/293,244 and 60/297,692 which were filed on May 24, 2001 and Jun. 21, 2001, respectively.

### BACKGROUND OF THE INVENTION

1. Field of the Invention

The present invention concerns an approach for immunoprevention and treatment of cancer in people who have higher risk of developing cancer or have developed cancer. Humans can be immunized by injection or oral administration to produce antibodies against N-glycolylneuraminic acid (anti-NeuGc) in blood circulation. The anti-NeuGc recognizes and binds to the specific antigen, NeuGc, which is present in various types of cancer. The anti-NeuGc level can be elevated by a vaccine, which comprises NeuGc-containing substance, in the form of injectable vaccination, pills, supplemented diet or nutritional supplement. This vaccination approach can be applied not only to those who are at high genetic or environmental risk of developing cancers but also to cancer patients who have undertaken conventional therapeutic treatment.

Chickens, similar to humans, do not naturally produce NeuGc in their bodies. However, NeuGc has been identified in lymphoma cells derived from Marek's virus infected chickens. The present invention also concerns an approach for immunoprevention of Marek's disease in chickens. Chickens can be immunized to produce antibodies against N-glycolylneuraminic acid (NeuGc) and maintain high titer in the eggs. The anti-NeuGc antibodies can bind specifically to several Marek's disease lymphomas cell lines but not to normal chicken tissues. Thus, feeding chickens with a diet containing NeuGc may elicit immune responses and result in anti-NeuGc antibodies accumulation in eggs. Chicks hatched from those eggs carry maternal antibodies, which should recognize Marek's disease infected cells at the early stage of infection and trigger host immune reactions. Therefore, chicks produced by this method are most likely to have immune resistance to the disease and capacity to suppress tumor growth.

2. Description of the Related Art

Distribution of NeuGc

Sialic acids are a group of acidic carbohydrates that are commonly found in nature (1). There are two most abundant forms of sialic acid: N-acetylneuraminic acid (NeuAc), which is ubiquitously present in nature, and N-glycolylneuraminic acid (NeuGc), which is present in most animals with the notable exception of humans and chickens (2). However, in humans, NeuGc has been identified by immunochemical and biochemical methods in breast cancer, lymphoma, gastric cancer, lung cancer, colon cancer, liver cancer, melanoma, and leukemia (3-5), as well as infectious diseases such as hepatitis, syphilis and leprosy (6). In chickens, NeuGc has been detected by using gas chromatography-mass spectrometry in gangliosides-rich fractions from five different chicken Marek's disease lymphomas cell lines (7).

NeuGc is synthesized in vivo from NeuAc with the addition of a single hydroxyl group by an enzyme called CMP-NeuAc hydroxylase (8). The gene encoding the enzyme has been cloned from mouse, chimpanzee and human(9). While mouse and chimpanzee gene codes for a functional enzyme, human gene has a partial deletion, resulting in a truncated form without the enzymatic activity(10). Interestingly, this is the only example, on genomic level, where a human gene is different from chimpanzee (11;12).

Thus, how is NeuGc synthesized in human cancerous cells if the responsible enzyme is missing? Although experimental evidence is lacking, one possibility is that human body may absorb NeuGc from food, resulting in its accumulation in fast growing cells like tumor cells.

Antibodies Against NeuGc

Antibody against Hanganutzin-Deicher antigen (anti-HD), which contains NeuGc as part of its immunogenic determinant, was initially identified from patients who had been exposed to animal sera, and was later detected in patients suffering various types of cancer, such malignant melanoma, colon cancer, breast cancer and leukemia (3;13) and some infectious diseases (14). Interestingly, Anti-HD level in cancer patient's serum seems to be inversely correlated to recurrence of cancer (15). In a study of patients with stage II melanoma, a significantly higher level of anti-HD was demonstrated in patients who were free of disease more than five years after surgery than in those who relapsed within two years. Thus, the antibody in patient's serum seems important in protecting against residual tumor or micrometastases. As another piece of supporting evidence, anti-HD was able to kill melanoma cells in an in vitro complement-dependent cytotoxicity assay (15).

Although it has been commonly accepted that healthy humans contain no or negligible level of HD antibody (16), our recent work (see Examples 1 and 2) indicates that antibodies against NeuGc (anti-NeuGc) is present in a majority of healthy individuals based on a sensitive flow cytometry assay (unpublished data, Zhu, A.). Anti-NeuGc has been purified from normal human sera by using affinity chromatography. Anti-NeuGc activity can be specifically inhibited by pre-incubation with NeuGc molecule, but not with NeuAc molecule. The anti-NeuGc antibody we identified in healthy people may be related to anti-HD reported in the literature.

Clinical Applications of Gangliosides

Gangliosides are common components of cell membranes. They are composed of a hydrophobic end buried in the membrane and a polysaccharide chain exposed to the external surroundings. Majority of sialic acids on the cell surface are part of polysaccharide chains of gangliosides. In fact, most work on HD antigen has been carried out using gangliosides as starting material (3;17;18).

Dramatic changes in gangliosides have been noted in tumors such as melanoma, neuroblastoma, and small cell lung cancers (19) (20). Following malignant transformation, normal skin melanocytes, which initially express mainly Gm3, begin to synthesize large amounts of Gd3, a different form of ganglioside. In addition, high levels of gangliosides have been detected in cancer patient's serum, which may play a role in suppressing host immune responses to the tumors.

A ganglioside vaccine has reached a phase III trial in melanoma patients. Patients who produced the antibodies had a significantly longer disease-free and overall survival than patients who showed no antibody response (21;22). Data from a clinical trial using ganglioside (N-Glycolyl-Gm3) vaccine in breast cancer patients showed that the vaccine was safe and immunologically effective. Considering the immunogenicity of a heterophile antigen and its expression in breast tumors, N-glycolylated ganglioside based therapeutic vaccines may be effective in the treatment of human breast cancer.

Antibodies against gangliosides have been identified in healthy individuals and their levels seem to decline with age (19). The observations may be of physiological significance considering that the antibodies can neutralize pathogens expressing abnormal gangliosides or gangliosides shed by cancerous cells. Thus, it is tempting to speculate that the decrease in anti-gangliosides with aging may contribute to higher susceptibility to cancer and other infections.

Immunoprevention of Cancer

In a recent review article titled “Immunoprevention of cancer: Is the time ripe?”, Forni G. et al. (23) pointed out that manipulation of the immune response to malignancy may provide a fresh and perhaps conclusive way of winning the long-lasting war against cancer. In fact, many experimental data suggest that the immunity induced by specific vaccination is much more effective in the inhibition of incipient tumors than in the cure of established tumors. As a tumor increases in size, it becomes refractory to immunotherapy. In other words, the efficacy of immunity declines as a tumor progresses. A study in mice indicated that a vaccination was effective even against poorly or apparently nonimmunogenic tumor challenge (24;25).

Immunoprevention of Marek's Disease in Chickens

Marek's disease is a viral lymphoproliferative disease, which is highly contagious and spreads mainly in young chicken flocks (26). Marek's Disease Virus (MDV) is ubiquitous, occurring in poultry-producing countries throughout the world. Even chickens raised under intensive production systems will inevitably suffer losses from the disease. MDV affects chickens from about 6 weeks of age, occurring most frequently between ages of 12 and 24 weeks and the mortality by this disease is extremely high.

Three forms of MD are clinically recognized: classical MD, acute MD and transient paralysis. Classical MD is characterized by peripheral nerve enlargement and paralysis is the dominant clinical sign. Mortality is variable but normally under 10-15 percent. In the acute form there are multiple and diffuse lymphomatous tumors in the visceral organs. An incidence of 10-30 percent is common in unvaccinated flocks and outbreaks involving up to 70% of the flock may be encountered. The pathological lesions in both classical and acute MD are essentially similar, involving the proliferation and infiltration of malignantly transformed T-lymphoblasts into normal tissues, peripheral nerves in the classical form and visceral organs in the acute form. Finally, the MDV has been shown to be responsible for encephalitis in young chickens characterized by sudden paralysis.

Since a method of treatment for MD is not available, the control of MD is almost entirely dependent on vaccination. Routinely, most chicks get vaccinated in the egg before they hatch. Thus they are born with special immunity to Marek's disease. If vaccinated after hatching, chicks require many days for immunity to take hold and thus are vulnerable in the meantime. Current vaccines comprise chemically inactivated virus vaccines or modified live-virus vaccines. However, inactivated vaccines require additional immunizations, disadvantageously contain adjuvants, are expensive to produce and are laborious to administer. Furthermore, some infectious virus particles may survive the inactivation process and may cause the disease after administration to the animal. In addition, vaccine resistant MDV strains may be produced due to mutations in MDV antigens (27).

Based on data from human studies, it is reasonable to hypothesize that if chickens have pre-existing anti-NeuGc in their blood circulation, they are more likely to be resistant to MDV infection and tumor development. Since chickens usually become infected at an early stage of development, it is thus desirable to build up the immune response at birth. We have shown that anti-NeuGc can be induced in hens by subcutaneous injection and accumulated in eggs (see Example 3). Thus, chicks hatched from eggs containing high titer of anti-NeuGc should carry maternal antibodies against NeuGc in their blood circulation and may become more resistant to MDV infection.

Although immune response can be achieved through a number of routes including subcutaneous injection or oral administration, oral administration is more desirable approach considering the vaccination of chickens must be done in a large scale.

It is, therefore, an object of the present invention to provide a novel approach to increasing anti-NeuGc level in human blood circulation in fighting against cancer and any other diseases bearing the specific antigen.

It is another object of the present invention to provide a procedure for triggering immune responses by oral administration or injection of NeuGc-containing substances, which include, but are not limited to, material derived from animal original or chemically synthesized.

It is another object of the present invention to provide a procedure for producing cells/molecules with high content of NeuGc residues, which are potent immunogens for inducing anti-NeuGc in humans.

It is a further object of the present invention to provide a simple procedure for efficiently measuring level of anti-NeuGc in human blood.

It is also an object of the present invention to provide a novel approach to increasing chicken's immune resistance to Marek's disease virus.

It is another object of the present invention to provide a procedure for producing eggs containing high titer anti-NeuGc antibody by oral immunization of chickens.

It is another object of the present invention to provide procedures both for preparing antibody extracts from egg yolk and for purifying anti-NeuGc antibody by affinity chromatography.

It is a further object of the present invention to provide a method for producing chicken supplemented food containing material that triggers anti-NeuGc antibody. The material includes pig brain or any other organs and cells containing NeuGc, in their original or processed form.

### SUMMARY OF THE INVENTION

Immunoprevention and Treatment of Cancer in Humans

Our work has led us to invent a novel approach to increasing human anti-NeuGc in blood circulation as a means of preventing and treating cancer and other diseases that carry the specific antigen.

Based on all the data above, we believe that if humans have high levels of anti-NeuGc in blood circulation, it is more likely for the human body to detect molecular alterations on the cell surface and thus initiate immune reactions. This is particularly important for individuals with the family history of cancer or living in cancer prone environment, although it can be used as adjuvant treatment for caner patients who have undertaken conventional therapeutic treatment.

A very similar scenario has been encountered for a well-studied antibody. An antibody against α 1,3-linked terminal galactose residue (anti-Gal) has been detected in most humans and constitutes between 1-5% of total naturally occurring antibodies in human blood (28;29). This galactose structure is widely present in animals except in humans and Old World monkeys such as chimpanzees and baboons because of the fact that the responsible enzyme (α1,3 galactosyltransferase) is mutated in these primates (8). The high level of anti-Gal is considered evolutionary advantageous because it constitutes the first immunological barrier of human bodies to intruding pathogens. In fact, hyperacute rejections commonly observed in xenotransplantation result mainly from the potent immune reactions between host anti-Gal and the α1,3-galactose antigens on donor cells/organs.

Based on the same rationale, genetic inactivation of CMP-NeuAc hydroxylase in humans may result in a second molecular marker that distinguishes humans from the rest of animal kingdom, serving as an additional means in human immune defense system. Like anti-Gal, anti-NeuGc antibody in human blood circulation may also play an important role in detecting and neutralizing foreign antigens. The observation that the level of anti-NeuGc seems much lower than that of anti-Gal could be explained by the fact that the mutation of CMP-NeuAc hydroxylase is a much recent event (6-7 million years ago) in the evolution than that of α1,3-galactosyltransferase (about 30-40 million years ago). From the evolutionary perspective, what we propose to do here may be merely to speed up what evolution would take care of by itself in the long run.

One of the concerns for using animal-derived materials as a vaccine is that the materials contain NeuGc antigen as well as other antigens, which are immunogenic to humans. To address this issue, it will be preferred to add NeuGc residues to human-derived cells or molecules. Using the modified cells/molecules as a vaccine, human immune system will thus only respond to the NeuGc antigen but not to any other part of cells/molecules.

There are a number of methods for such modification. As shown in FIG. 9, human cells (or molecules including glycoproteins and gangliosides) can be incubated with sialyltransferase using CMP-NeuGc as a substrate for 2 hours at 37 C. Alternatively, cells can be pre-treated with neuraminidase to remove NeuAc from the cell surface to create more sites for adding NeuGc in the sialyltransferase reaction. CMP-NeuGc can be produced from CMP-NeuAc with CMP-NeuAc hydroxylase that has been cloned from mouse and chimpanzee.

A second method is illustrated in FIG. 10. Fresh peripheral blood is taken from individuals (preferably blood type O, Rh−) for an ex vivo culture, where hematopoietic stem cells differentiate into mature red cells in the presence of cytokines such as I1-3, F1, K1 and EPO. A vector carrying CMP-NeuAc hydroxylase cDNA will be used in transfection of the progenitor cells. The expression of the enzyme in differentiating cells will result in the expression of NeuGc on the surface of mature red cells, which can be accurately measured by purified human anti-NeuGc in flow cytometry. Human red cells carrying NeuGc can thus be used as a potent vaccine to trigger anti-NeuGc in human recipients.

In addition, mammalian cells can be manipulated to over-express NeuGc by adding synthetic precursor such as N-glycolylmannosamine pentaacetate to the tissue culture (30).

Furthermore, NeuGc-rich gangliosides may be directly incorporated into membrane bilayer of mammalian cells (See Example 7). Cells thus generated may provide antigens with enhanced immunogenicity (See Example 8).

The approach of immunoprevention is most advantageous if a vaccine is against a number of diseases rather than against a single target. Indeed, an increased anti-NeuGc in blood circulation by immunization will function as surveillance for NeuGc antigen associated with cell malignancy and intruding pathogens. High levels of anti-NeuGc can be potentially beneficial to one's health by increasing the resistance to infection and disease. Therefore, our procedure can be applied, at a large scale, not only to people with a high genetic or environmental risk of developing cancers, but also to the general population as well.

Immunoprevention of Marek's Disease in Chickens

Our work has led us to invent a novel approach to increasing chicken's immune resistance to Marek's disease virus, the most serious disease threat to the poultry industry. Our approach comprises three steps. First, chickens will be fed with food containing antigen that induces host immune responses. Then immunized chickens will lay eggs that contain specific antibody against NeuGc. Finally, chicks hatched from those eggs will carry the same antibodies and are most likely to be resistant to MDV infection.

The most common and traditional method for immunizing chickens is by subcutaneous injection of antigen alone or in combination with an adjuvant. A far more efficient and economic way of immunization, particularly for large-scale immunization, is by oral administration. It would be a tremendous savings in cost and labor for large-scale immunization if chickens can be handled via supplemented diet. We have identified a NeuGc-containing material as an ingredient in the supplemented food. Pig brain is an extremely rich source of NeuGc and is readily available. Fresh or frozen pig brain can be ground and lyophilized. The processed tissue can be stored in a dry state and used directly for supplemented diet. However, although we use pig brain as an example here, the material that can be added to the diet includes, but is not limited to, other organs, blood products and tissue extracts (from any animals) provided the NeuGc-containing glycoconjugates are present. In addition, mammalian cells can be manipulated to over-express NeuGc by adding synthetic precursor such as N-glycolylmannosamine pentaacetate to the tissue culture (30). This procedure may generate substance with enhanced immunogenicity.

Egg-laying hens can be fed with the supplemented diet containing small amount of processed pig brain (les than 1% of regular diet by weight) for an extensive period of time (between 2-3 weeks). Eggs can be collected for measuring anti-NeuGc titer in egg white as an indication of the host immune responses. Chicks hatched from eggs containing high titer of anti-NeuGc should carry maternal antibodies against NeuGc in their blood circulation. By binding to MDV tumors expressing NeuGc on the cell surface, anti-NeuGc antibodies are likely to provide the chickens with immune suppression and resistance to MDV.

The various features of novelty, which characterize the invention, are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

FIG. 1. Flow cytometry analysis of human anti-nonαGal. The antibodies were incubated with pRBC (type O) at room temperature for 30 min. Anti-human IgG (H+L) conjugated with R-phycoerythrin (PE) was used as a second antibody. (A) The antibodies were bound to untreated (peak 1) or sialidase-treated (peak 2) pRBC. In an inhibition assay, the antibodies were preincubated with NeuAc (peak 3) or NeuGc (peak 4) prior to binding with untreated pRBC. Cells plus the secondary antibody (peak 5) was used as background controls for the flow cytometry. (B) The antibodies were pre-incubated with increasing concentrations of NeuGc (●) or NeuAc (∘) prior to flow cytometry analysis. The percentage of binding (% MFI) was plotted against sialic acid concentrations.

FIG. 2. Distribution of xenoreactive antibodies in healthy human volunteers by hemagglutination assay. Untreated pRBC was used for measuring the total xenoreactive antibodies in the serum. α-Galactosidase-treated pRBC was used for measuring xenoreactive antibodies minus anti-αGal. Anti-NeuGc was characterized by pre-incubation with NeuGc followed by hemagglutination with α-galactosidase-treated pRBC. Thus, the relative levels of anti-αGal and anti-NeuGc in 19 sera are demonstrated as gray and black bars, respectively, whereas the white bar represents undefined xenoreactive antibodies.

FIG. 3. Flow cytometry analysis of chicken antibody raised against porcine gangliosides. The antibody sample was incubated with α-galactosidase pRBC (type O) at room temperature for 0.5 hour. Anti-chicken IgG (H+L) conjugated with FITC was used as a second antibody. Cells alone or cells plus the secondary antibody (1) were used as a background control for the flow cytometry (Becton Dickinson). Chicken antibodies (0.1 ul each) prepared from pre-immune eggs (2) and post-immune eggs (3) were analyzed by flow cytometry analysis. Post-immune antibodies were pre-incubated with 3.75 mM NeuGc (4) or 3.75 mg/ml porcine gangliosides (5) prior to the analysis.

FIG. 4. Affinity purification of chicken antibodies. Antibodies from various steps of purification were analyzed by flow cytometry, with (1) as control (no antibody). (A) Chicken antibodies prepared from pre-immune eggs (2) and post-immune eggs (3) were individually applied to thyroglobulin-agar column and antibodies bound to the column were eluted with glycine-HCl, pH2.5. (B) Binding capacity of the affinity column was examined. To a 3 ml thyroglobulin column, chicken antibody (1 ml each time, total 4 ml) was loaded and unbound fraction was collected. (2) is the sample loaded to the column. (3) through (6) are the unbound fractions from the column after each loading.

FIG. 5. Selective inhibition of chicken anti-NeuGc and anti-gangliosides. (A) Prior to flow cytometry analysis, both antibodies were pre-incubated with increased amount of NeuGc. (B) Both antibodies were pre-incubated with increasing amount of porcine gangliosides.

FIG. 6. Immunization of chickens by oral administration. Chickens were divided into three groups as described in the text: Group 1: H4-1, H4-3 and H4-6 (except H4-2 that died in a natural cause during the study); Group 2: H2-1, H2-2 and H2-4 (except H2-3 that produce produces eggs with unusually high lipid contents); and Group 3: H3-1, H3-2, H3-3 and H3-4. Antibody levels in eggs were measured in a standard ELISA. The immune response in the diagram is expressed as the ratio of ELISA values derived from post-immune eggs over pre-immune eggs.

FIG. 7. Incorporation of porcine gangliosides into human red cells. Different concentrations of porcine gangliosides were incubated with human red cell at room temperature overnight. The treated red cells were washed three times with PBS and then tested for binding with purified chicken anti-NeuGc (10 μl) with (∘) or without (●) free gangliosides as an inhibitor.

FIG. 8. Immunization of chickens with gangliosides-incorporated autologous RBC. Following the described procedure for ganglioside incorporation, chicken RBC (#236) was completely lysed due to excess amount of gangliosides. Thus, the RBC stroma was collected for subcutaneous injection of chicken (#236). On the other hand, chicken RBC (#245) was intact after gangliosides incorporation and was intravenously injected into chicken (#245). The arrows indicate the initial immunization and following-up boosting. The immune responses were tested using chicken sera in a standard ELISA.

FIG. 9. Enzymatic reactions for generating NeuGc residues on human cell surface (or any other cells and molecules that do not express NeuGc residues). Human cells such as RBC can be directly incubated with sialyltransferase using CMP-NeuGc as a substrate for 2 hours at 37 C. Alternatively, cells can be pre-treated with neuraminidase to remove NeuAc from the cell surface to create more sites for adding NeuGc in the sialyltransferase reaction. CMP-NeuGc can be produced from CMP-NeuAc with CMP-NeuAc hydroxylase that has been cloned from mouse and chimpanzee.

FIG. 10. Expression of NeuGc residues on human red cell surface in an ex vivo culture system. Fresh peripheral blood is taken from individuals (preferably blood type O, Rh−) for an ex vivo culture, where hematopoietic stem cells differentiate into mature red cells in the presence of cytokines such as I1-3, F1, K1 and EPO. A vector carrying CMP-NeuAc hydroxylase cDNA can be used in transfection of the progenitor cells. The expression of the enzyme in differentiating cells will result in the expression of NeuGc on the surface of mature red cells, which can be directly measured by purified anti-NeuGc in flow cytometry.

### DETAILED DESCRIPTION OF THE PRESENTLY PREFERRED EMBODIMENTS

### EXAMPLE 1

Identification of anti-NeuGc from Healthy Human Sera

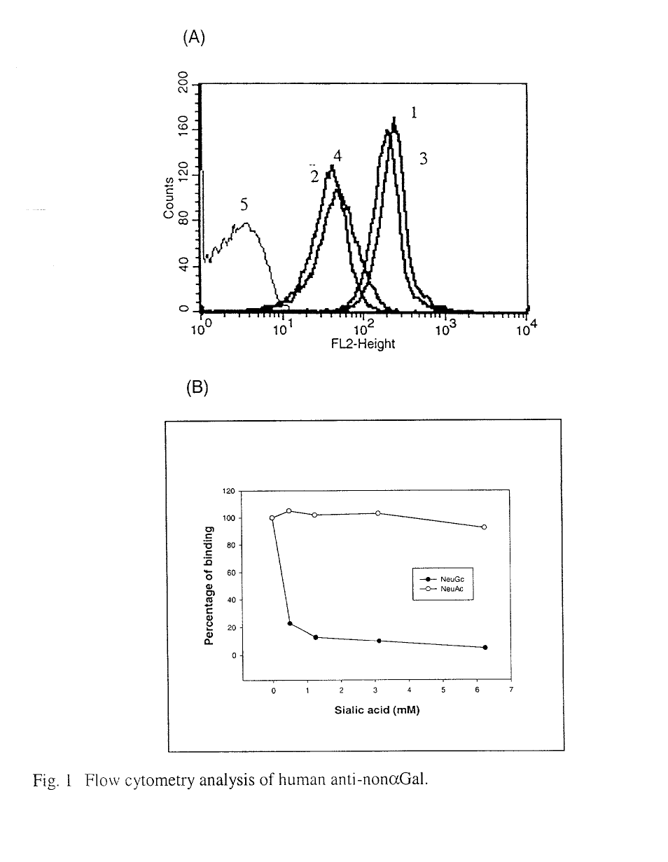

Human anti-nonαGal was prepared by depleting anti-αGal from the total xenoreactive antibodies in the serum (31). The first hint that anti-nonαGal may bind to cell surface NeuGc residues was based in the observation that pre-treatment of porcine red blood cells (pRBC) with sialidase resulted in a significant decrease in antibody binding (FIG. 1A). On the other hand, pre-treatment of fresh pRBC with a number of other exoglycosidases (β-galactosidase, α-N-acetylgalactosaminidase and hexosaminidase) had no effect on antibody binding (data not shown). To elucidate the nature of antigens recognized by anti-nonαGal, the antibodies were preincubated with 10 mM NeuGc or NeuAc prior to the binding with pRBC. Although these two compounds only differ from each other by one hydroxyl group, NeuGc exhibited a strong inhibitory effect on antibody binding to the cells (peak 4) whereas NeuAc had no effect (peak 3) under the same conditions (FIG. 1A).

When the percentage of binding was plotted against the amount of sialic acid as an inhibitor (FIG. 1B), the antibody binding dropped significantly with the increase in NeuGc. Ninety percent of inhibition was observed at 1.2 mM of NeuGc. On the other hand, there was essentially no inhibition with NeuAc as high as 6 mM. Furthermore, based on mean fluorescent intensity (MFI), the I₅₀ value (the concentration of NeuGc that gives rise to 50% inhibition) was determined to be approximately 0.073 mM. These data strongly suggest that the majority (˜80% based on MFI values) of anti-nonαGal we isolated from pooled type AB sera are directed against carbohydrate antigens containing terminally-linked NeuGc, and can thus be designated as anti-NeuGc.

### EXAMPLE 2

Distribution of anti-NeuGc in Healthy Human Population

In order to directly measure the level of anti-NeuGc in healthy human sera using flow cytometry analysis, the binding of human sera with α-galactosidase-treated pRBC was examined in the presence or absence of 7.5 mM of NeuGc. While treatment of pRBC with α-galactosidase eliminates binding of anti-αGal, a reduction in MFI value in the presence of NeuGc would suggest anti-NeuGc activity. Under these conditions, anti-NeuGc activity was detected in 17 out of 20 sera from healthy volunteers, suggesting an 85% incidence of anti-NeuGc-positive sera in the healthy population.

Applying the same strategy to hemagglutination assays, the amounts of anti-NeuGc in normal human sera were quantitatively compared with those of anti-αGal, which have been well characterized. The data in FIG. 2 indicates that anti-NeuGc activity was detected in 17 out of 20, or 85% of normal human volunteers, in agreement with the data from flow cytometry analysis. The level of anti-NeuGc in serum varies among different individuals and, though less than anti-αGal, anti-NeuGc constitutes a significant portion of human xenoreactive antibodies.

### EXAMPLE 3

Immunization and Purification of anti-NeuGc in Chickens

Porcine gangliosides, a rich source of terminally linked NeuGc residues, were used as antigen to immunize chickens. Two hens (H71 and H72) were immunized at multiple subcutaneous sites with the antigen, together with complete Freund's adjuvant. After two boosts, chicken eggs were collected and antibodies, IgY (equivalent to mammalian IgG), were prepared from egg yolk according to the published procedure. Briefly, after removing egg white, diluted yolk was extracted with chloroform. The upper aqueous phase was recovered and precipitated with a two-step polyethylene glycol process. However, the procedure is time-consuming and requires extra care for the use of organic solvent. Therefore, we worked out a modified procedure, which eliminates chloroform and makes feasible both small and large-scale antibody preparations. Using this procedure, we routinely obtain 7-10 mg IgY per ml of egg yolk (15-20 ml yolk per egg). SDS-PAGE analysis revealed that the antibody preparation contains mainly two bands corresponding to immunoglobulin light and heavy chains.

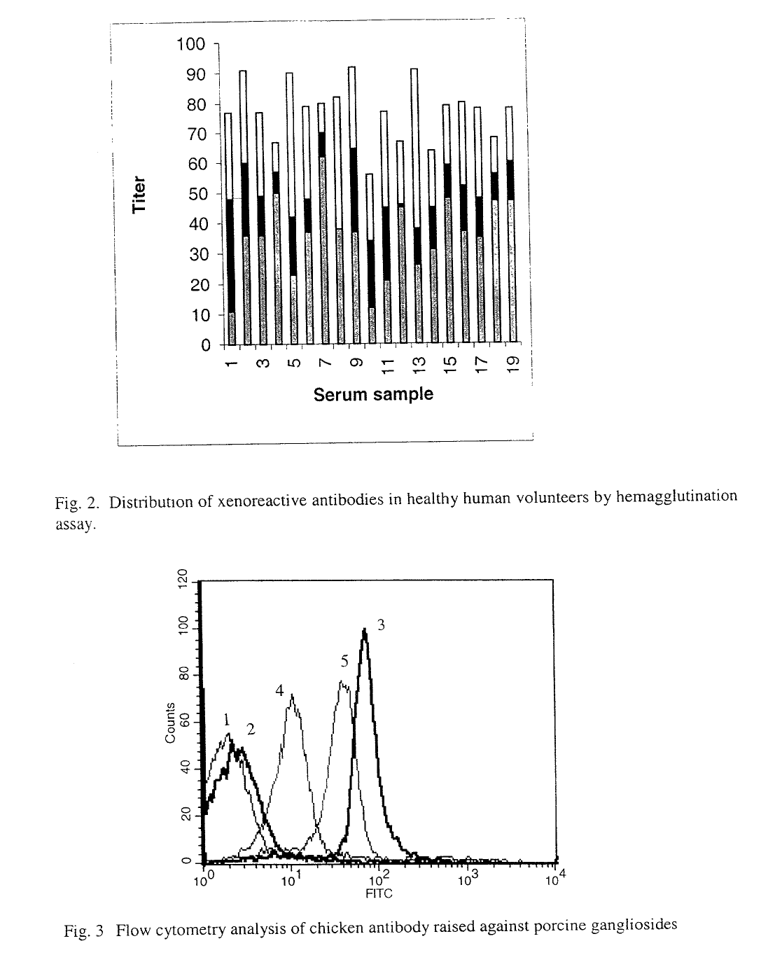

To examine the effect of ganglioside immunization and level of induced antibodies in eggs, IgY prepared from pre-immune eggs and post-immune eggs were analyzed by flow cytometry analysis using α-galactosidase-treated pRBC. As shown in FIG. 3, while pre-immune IgY (2) essentially did not bind the pRBC, the post-immune IgY (3) showed a strong binding activity. Furthermore, this binding activity can be partially inhibited by pre-incubation of the antibodies with NeuGc (4) or porcine gangliosides (5), thus confirming the binding specificity of the raised antibodies. Both hens (H71 and H72) responded equally well toward immunization. We are collecting 20-25 eggs per hen per month. Over four months after immunization, we can still detect significant amount of antibody activity, although the titer has slightly dropped from its peak.

Our data showed that chickens do not have pre-existing anti-NeuGc, suggesting that under normal conditions chicken feeds do not contain substances that would trigger the immune responses.

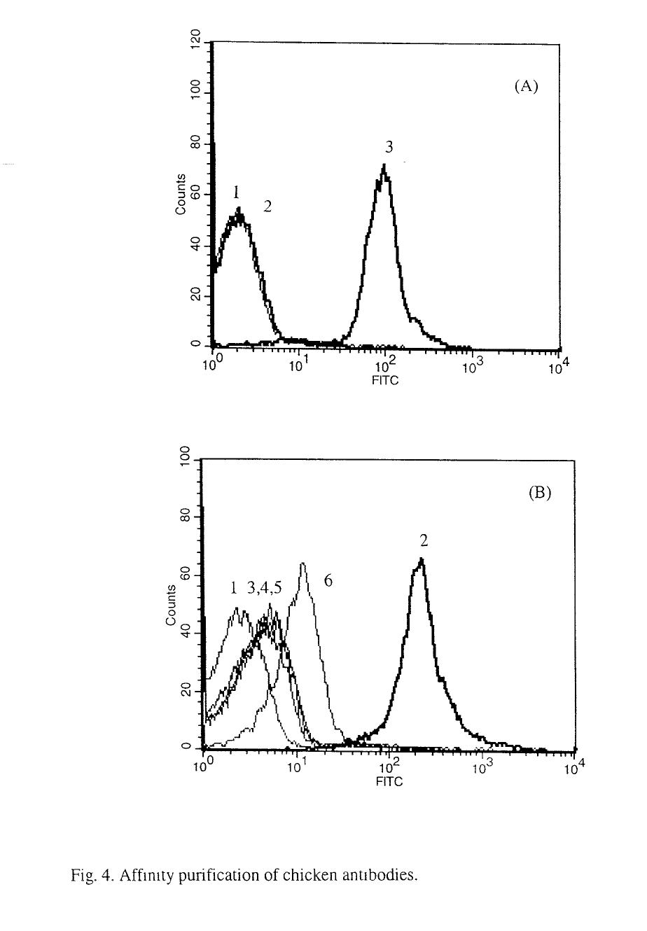

In order to explore the possibility of using thyroglobulin column to further purify chicken antibodies, we first loaded post-immune IgY to the column. After removing unbound fraction, the bound faction was eluted with glycine-HCI, pH 2.5. As a control, the same procedure was repeated with pre-immune IgY. As shown in FIG. 4A, the post-immune IgY fraction (3) eluted with the low pH buffer demonstrated significant activity, whereas pre-immune IgY (2) failed to recognize the thyroglobulin resin. In order to examine the specificity and capacity of the column, we loaded 1 ml of chicken antibodies to the column (3 ml volume) and collected the unbound fraction. The procedure was repeated for three more times (totally 4 ml of antibodies loaded). As shown in FIG. 4B, for the first three rounds (3 through 5) almost all specific antibodies bound to the column in comparison to the loading sample (2). In the fourth round (6), slightly more antibody activity was detected in the unbound fraction, suggesting the saturation of the column. Thus, we conclude that under these conditions the specific antibodies of interest can be all bound to the column, with the binding capacity of approximately 1 ml chicken antibodies per ml resin.

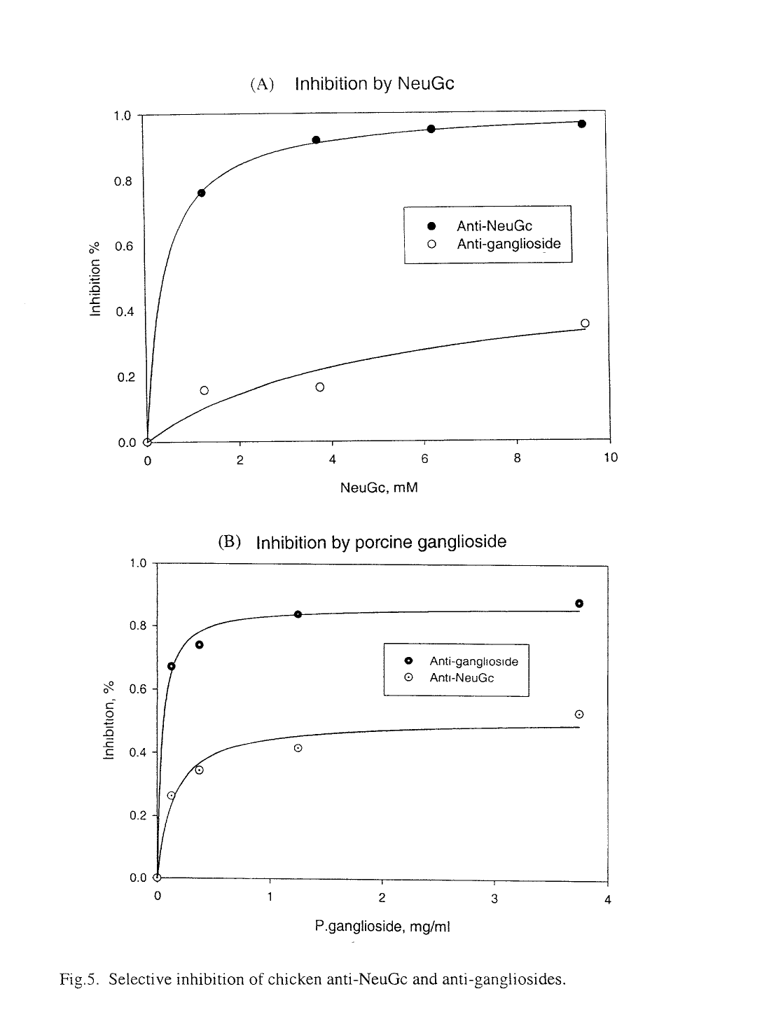

To further purify chicken antibodies that bind to the thyroglobulin column, we eluted the column with PBS containing NeuGc, followed by glycine-HCI, pH 2.5. The two resultant fractions are designated as anti-NeuGc and anti-gangliosides, respectively, based on detailed characterization of their binding specificities as described below. Quantitatively, there are roughly ten times more anti-NeuGc than anti-gangliosides activity in the chicken antibodies based on flow cytometry analysis (assuming there are the same amounts of binding sites for both antibodies on pRBC). Both of antibody fractions, anti-NeuGc and anti-gangliosides, derived from further purification of the post-immune IgY bind to pRBC in flow cytometry. However, their binding can be distinctively inhibited with NeuGc and porcine gangliosides. As shown in FIG. 5A, the binding of anti-NeuGc was rapidly inhibited with an increasing concentration of NeuGc, illustrating a typical saturation curve. At 9.5 mM of NeuGc, more than 95% of inhibition of anti-NeuGc was observed. On the other hand, pre-incubation of NeuGc with anti-gangliosides only modestly inhibited the antibody binding, with a maximal inhibition of approximately 30%. When porcine gangliosides were used as inhibitor for antibody binding, a reverse response was revealed. As shown in FIG. 5B, anti-gangliosides were inhibited by gangliosides more effectively than anti-NeuGc was. Based on these data, we suggest that the affinity procedure we applied for the isolation of anti-NeuGc and anti-gangliosides divided the chicken antibodies of interest into two groups of antibodies with distinctive, but slightly overlapped binding specificities.

### EXAMPLE 4

Detection of NeuGc Antigen in Cancer Tissue Samples and a Cancer Cell Line

Tissue samples (breast cancer, colon cancer and gastric cancer) are prepared according to a standard procedure: From paraffin-embedded tissue blocks, 4-um thick sections are cut, deparaffinized and dehydrated in a standard manner. Endogenous peroxidase is blocked by incubating sections with 0.3% hydrogen peroxide for 30 minutes at room temperature. After rinsing three times with PBS, the sections are incubated with 1% ovalbumin in PBS for 30 min, followed by incubating with chicken anti-NeuGc antibody (1:200 dilution). Specific binding was then detected by using anti-chicken antibody conjugated with biotin and streptavidin-horse radish peroxidase.

Under these conditions, we observed NeuGc-positive samples as follows: 40% breast cancer, 36.6% colon cancer and 43.3% gastric cancer.

More recently, we have succeeded in detecting NeuGc antigen in breast cancer cell line MCF-7 using purified chicken anti-NeuGc antibody. Cells are seeded in chambered slides under cell culture conditions. After growing upto 80%-90% confluence (approximately 2-3 days), cells are gently washed with PBS, pH 7.2, and then fixed in 300 ul methanol for 5 min at room temperature. The chambered slides are blocked with PBS containing 2% BSA prior to the incubation with purified chicken anti-NeuGc in a ECL fluorescence immunoassay. Under these condition, we observed the binding of MCF-7 cells with anti-NeuGc at 1:500 fold dilution. Its binding specificity was confirmed by inhibition with 30 mM NeuGc.

### EXAMPLE 5

Immunization of Chickens by Oral Administration

Objective of the experiment was to induced immune responses in chickens by oral administration. 450 g of pig brain were collected from 4 pigs (300-400 pounds for each pig) and frozen for storage and shipping. The tissue was ground and lyophilized, resulting in 85 g in powder. 40 g of pig brain powder and 10 pounds of chicken feed (Highland complete layer) were sent to a processing manufacture (Bio-Serve Inc., One 8th St. Frenchtown, N.J. 08825). They ground everything together and made pellet again. Thus, the supplemented feed will be in the same shape as the original.

12 egg-laying hens were divided into 3 group. Group 1 is the control, fed with the regular food (Highland complete layer). Group 2 is fed with 25% supplemented food/75% regular food (1 g of pig brain powder/pounds of food). Group 3 is fed with 1.25% supplemented food/98.75% regular food (0.05 g of pig brain powder/pounds of food). After feeding different diet in the three groups for one month, all the hens were then fed with the regular food.

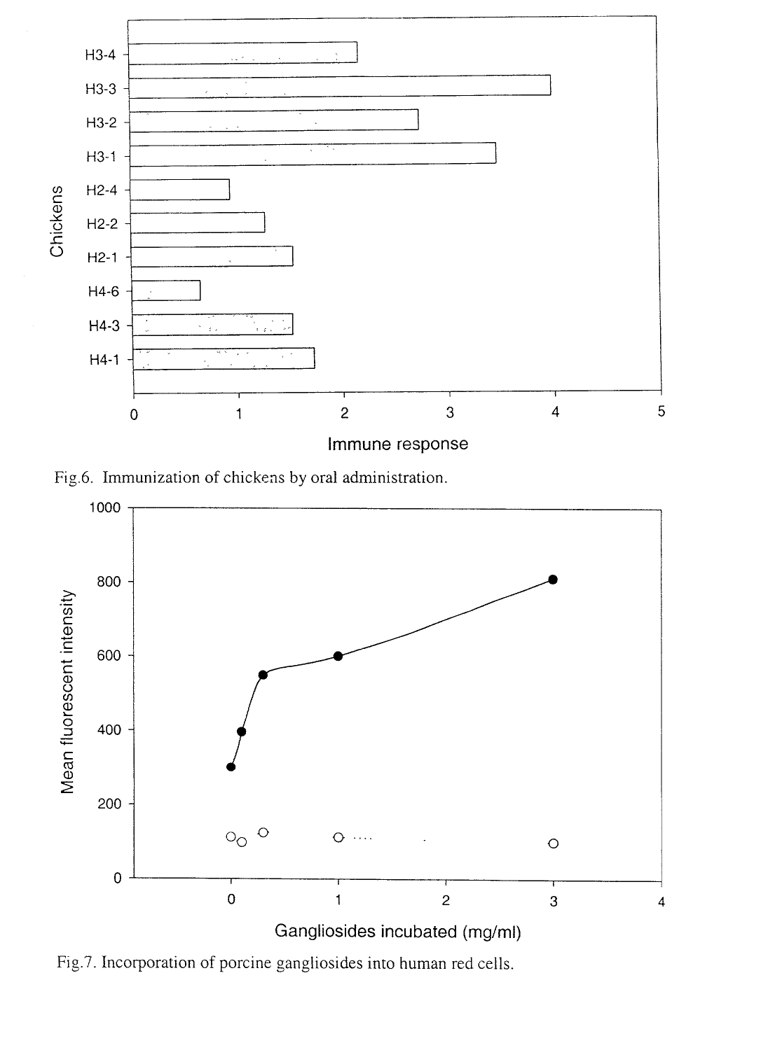

Although each group had 4 chickens in the study, one chicken in Group 1 died and one chicken in Group 2 had unusually high lipid level. Therefore, the two chickens were excluded from the final analysis. As shown in FIG. 6, Group 1: H4-1, H4-3 and H-4-6; Group 2: H2-1, H2-2 and H2-4; and Group 3: H3-1, H3-2, H3-3 and H3-4. Furthermore, every chicken in Group 3 has a significant increase in the immune response, whereas the immune response in Group 2 is not distinguishable from Group 1. Therefore, Inducing anti-gangliosides level in chickens by oral administration is a feasible approach. The amount of antigen added to the feed is important for the immune responses, although excess amount of the supplement in the food (as the case in Group 3) may suppress the immune response.

### EXAMPLE 6

Preparation of Gangliosides from Porcine Red Blood Cells (RBC)

1. Collect porcine RBC in EDTA

2. Wash RBC with PBS, ×3

3. Lyse RBC with 10 volume of 0.2% HAc

4. Wash the RBC stroma with water, ×3, 2600 g, for 15 min

5. After treatment with a large volume of acetone, the dried stroma is collected by filtration

6. The acetone-dried stroma is swelled and homogenized with 4 volume of 0.01M KCl

7. Add 4 volume of tetrahydrofuran, stirring O/N, RT

8. After filtering through a Buchner funnel, the residue is re-extracted 3 times with tetrahydrofuran:water (8:1)

9. The combined filtrate is dried in a rotary evaporator

10. Add 0.6 N NaOH in methanol (the volume equals the weight of the acetone-dried stroma), 37 C, 6 hr

11. After extensive dialysis, the content is dried in a rotary evaporator.

12. Individual gangliosides can be further purified by HPLC.

13. The isolated gangliosides are analyzed by TLC, TLC-immunoplotting or GC-MS.

### EXAMPLE 7

Direct Incorporation of Porcine Gangliosides into Human RBC

In order to demonstrate the incorporation of NeuGc-containing gangliosides RBC membranes, we mixed human RBC with different amounts of porcine gangliosides. After incubating at room temperature overnight, the treated RBC were washed three times with PBS and then tested for binding with purified chicken anti-NeuGc (10 μl) with (∘) or without (●) free gangliosides as an inhibitor. As shown in FIG. 7, gangliosides were indeed inserted into red cell membranes and the binding with anti-NeuGc was inhibited by free gangliosides. Furthermore, the incorporated gangliosides seem stable in the lipid bilayer since we did not notice any significant decrease in antibody binding with treated red cells that had been stored in PBS for 5 days (data not shown).

### EXAMPLE 8

Chickens Immunized with NeuGc-rich Gangliosides-incorporated RBC

Procedure

Preparation of gangliosides-incorporated RBC (under sterilized conditions)

1. Wash chicken RBC with PBS, pH7.4, ×3 (1,500 rpm, 3 min) and make 10% suspension.

2. Prepare the following:

| No. | Chicken | 10% RBC | Gangliosides | PBS |
|---|---|---|---|---|
| 1 | #236 | 1 ml | Porcine RBC | 9 ml |
| | | | gangliosides | |
| 2 | #245 | 1 ml | Porcine brain | 9 m. |
| | | | gangliosides | |

3. O/N incubation at RT.

Observation: few lysis in No. 2 but complete lysis in No. 1.

4. Wash No. 2 RBC with PBS, ×3 (1500 rpm, 5 min).

5. Wash No. 1 RBC stroma, ×3 (5,000 rpm, 5 min).

6. Resuspend RBC in 1 ml PBS (10%).

7. Sample No. 1 was immunized for chicken #236 (subcutaneous injection), and No. 2 for chicken #245 ( intravenous injection).

8. Chicken sera were analyzed for anti-gangliosides antibodies by ELISA.

Results:

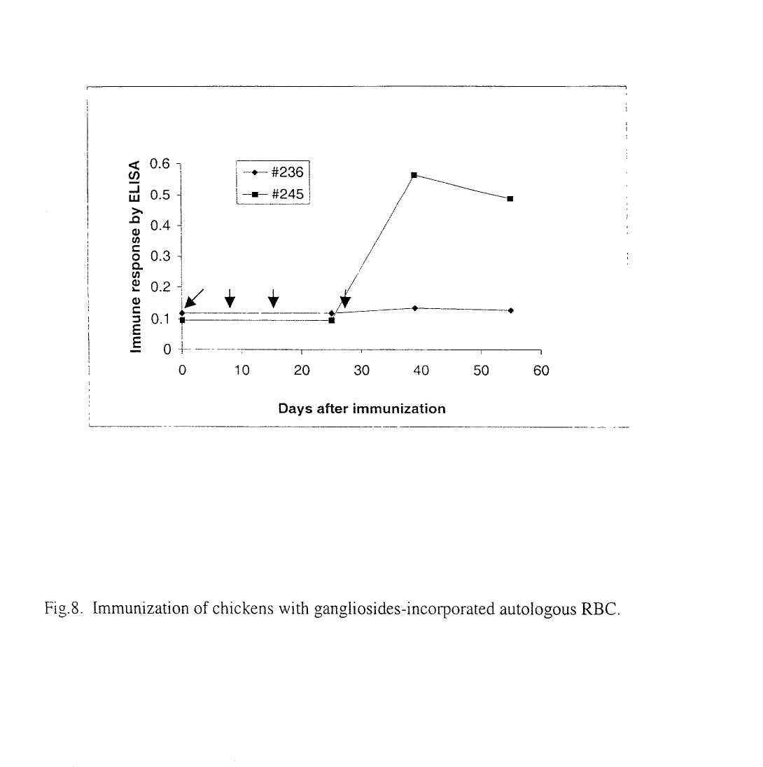

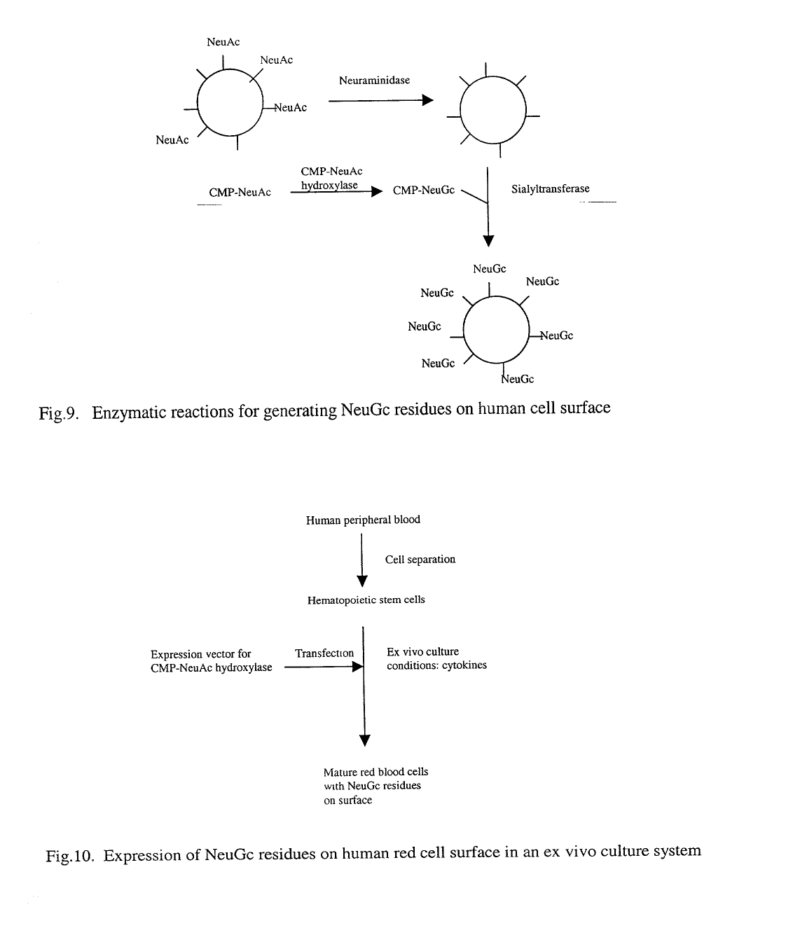

After primary immunization followed by three boosts, chicken #245 had a significant increase in antibody titer and remained high by day 55 (FIG. 8). On the other hand, chicken #236 that was s.c. injected with RBC stroma did not induce immune response, as shown in FIG. 8. Our data indicate that it is feasible to introduce immune response by using gangliosides-incorporated autologous RBC as immunogen even without using any adjuvants. However, in order to assess the efficacy of this immunization approach in a statistically significant manner, more animals would be needed in the study.

In addition, based on such a study, we will be able to extrapolate the amount of gangliosides-incorporated RBC needed for humans, as well as treatment schedule for human immunization.

The invention is not limited by the embodiments described above which are presented as examples only but can be modified in various ways within the scope of protection defined by the appended patent claims.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto. All cited references are hereby incorporated by reference in their entirety.

References

1. Schauer R. Sialic acids, chemistry, metabolism, and function. Wien: Springer-Verlag, 1982.

2. Gottschalk A. Glycoproteins; their composition, structure and function. Amsterdam: Elsevier Pub. Co., 1972.

3. Higashi H, Nishi Y, Fukui Y et al. Tumor-associated expression of glycosphingolipid Hanganutziu-Deicher antigen in human cancers. Gann 1984:75: 1025-1029.

4. Hirabayashi Y, Kasakura H, Matsumoto M et al. Specific expression of unusual GM2 ganglioside with Hanganutziu-Deicher antigen activity on human colon cancers. Jpn J Cancer Res 1987:78: 251-260.

5. Kawai T, Kato A, Higashi H, Kato S, Naiki M. Quantitative determination of N-glycolylneuraminic acid expression in human cancerous tissues and avian lymphoma cell lines as a tumor-associated sialic acid by gas chromatography-mass spectrometry. Cancer Res 1991:51: 1242-1246.

6. Nishimaki T, Kano K, Milgrom F. Hanganutziu-Deicher antigen and antibody in pathologic sera and tissues. J Immunol 1979:122: 2314-2318.

7. Naiki M, Fujii Y, Ikuta K, Higashi H, Kato S. Expression of Hanganutziu and Deicher type heterophile antigen on the cell surface of Marek's disease lymphoma. Adv Exp Med Biol 1982:152: 445-456.

8. Shaw L, Schauer R. Detection of CMP-N-acetylneuraminic acid hydroxylase activity in fractionated mouse liver. Biochem J 1989:263: 355-363.

9. Kawano T, Koyama S, Takematsu H et al. Molecular cloning of cytidine monophospho-N-acetylneuraminic acid hydroxylase. Regulation of spe. J Biol Chem 1995:270: 16458-16463.

10. Irie A, Suzuki A. CMP-N-Acetylneuraminic acid hydroxylase is exclusively inactive in humans. Biochem Biophys Res Commun 1998:248: 330-333.

11. Brinkman-Van der Linden E C, Sjoberg E R, Juneja L R, Crocker P R, Varki N, Varki A. Loss of N-glycolylneuraminic acid in human evolution. Implications for sialic acid recognition by siglecs. J Biol Chem 2000:275: 8633-8640.

12. Chou H H, Takematsu H, Diaz S et al. A mutation in human CMP-sialic acid hydroxylase occurred after the Homo-Pan divergence. Proc Natl Acad Sci USA 1998:95: 11751-11756.

13. Kawachi S, Saida T. Analysis of the expression of Hanganutziu-Deicher (HD) antigen in human malignant melanoma. J Dermatol 1992:19: 827-830.

14. Morito T, Kano K, Milgrom F. Studies on the Paul-Bunnell antigen-antibody system. III. Detection of a Paul-Bunnell-related antigen in syphilis and leprosy. Int Arch Allergy Appl Immunol 1983:71: 117-121.

15. Nakarai H, Chandler P J, Kano K, Morton D L, Irie R F. Hanganutziu-Deicher antigen as a possible target for immunotherapy of melanoma. Int Arch Allergy Appl Immunol 1990:91: 323-328.

16. Tachi Y, Kobayashi T, Yokoyama I et al. Variability of cytotoxicity to pig cultured cells and its determinant factor in human sera. Transplant Proc 1998:30: 71-73.

17. Higashi H, Sasabe T, Fukui Y, Maru M, Kato S. Detection of gangliosides as N-glycolylneuraminic acid-specific tumor-associated Hanganutziu-Deicher antigen in human retinoblastoma cells. Jpn J Cancer Res 1988:79: 952-956.

18. Merrick J M, Zadarlik K, Milgrom F. Characterization of the Hanganutziu-Deicher (serum-sickness) antigen as gangliosides containing n-glycolylneuraminic acid. Int Arch Allergy Appl Immunol 1978:57: 477-480.

19. Ravindranath M H, Gonzales A M, Nishimoto K, Tam W Y, Soh D, Morton D L. Immunology of gangliosides. Indian J Exp Biol 2000:38: 301-312.

20. Wagener R, Rohn G, Schillinger G, Schroder R, Kobbe B, Ernestus R I. Ganglioside profiles in human gliomas: quantification by microbore high performance liquid chromatography and correlation to histomorphology and grading. Acta Neurochir (Wien) 1999:141: 1339-1345.

21. Livingston P, Zhang S, Adluri S et al. Tumor cell reactivity mediated by IgM antibodies in sera from melanoma patients vaccinated with GM2 ganglioside covalently linked to KLH is increased by IgG antibodies. Cancer Immunol Immunother 1997:43: 324-330.

22. Yao T J, Meyers M, Livingston P O, Houghton A N, Chapman P B. Immunization of melanoma patients with BEC2-keyhole limpet hemocyanin plus BCG intradermally followed by intravenous booster immunizations with BEC2 to induce anti-GD3 ganglioside antibodies. Clin Cancer Res 1999:5: 77-81.

23. Forni G, Lollini P L, Musiani P, Colombo M P. Immunoprevention of cancer: is the time ripe? Cancer Res 2000:60: 2571-2575.

24. Musiani P, Modesti A, Giovarelli M et al. Cytokines, tumour-cell death and immunogenicity: a question of choice. Immunol Today 1997:18: 32-36.

25. Dranoff G, Mulligan R C. Gene transfer as cancer therapy. Adv Immunol 1995:58: 417-454.

26. Kreager K S. Chicken industry strategies for control of tumor virus infections. Poult Sci 1998:77: 1213-1216.

27. Witter R L. Control strategies for Marek's disease: a perspective for the future. Poult Sci 1998:77: 1197-1203.

28. Cooper, D K C. Xenoantigesn and xenoantibodies. Xenotransplantation. 5, 6-17. 1998. Ref Type: Generic

29. Galili U, Shohet S B, Kobrin E, Stults C L, Macher B A. Man, apes, and Old World monkeys differ from other mammals in the expression of alpha-galactosyl epitopes on nucleated cells. J Biol Chem 1988:263: 17755-17762.

30. Collins B E, Fralich T J, Itonori S, Ichikawa Y, Schnaar R L. Conversion of cellular sialic acid expression from N-ac. Glycobiology 2000:10: 11-20.

31. Zhu A. Binding of human natural antibodies to nonalphaGal xenoantigens on porcine erythrocytes. Transplantation 2000:69: 2422-2428.

I claim:

1. A method of increasing anti-NeuGc antibody level in the blood of an animal comprising administrating to the animal a substance containing NeuGc.

2. The method of claim 1 wherein the animal is a human.

3. The method of claim 2 wherein the human has a higher risk of developing cancer or has developed cancer.

4. The method of claim 3 wherein the cancer is selected from the group consisting of breast cancer, lymphoma, stomach cancer, lung cancer, colon cancer, liver cancer, melanoma, and leukemia.

5. The method of claim 1 wherein the animal is a bird.

6. The method of claim 5 wherein the bird is a chicken.

7. The method of claim 6 wherein the chicken is at risk of developing Marek's Disease.

8. The method of claim 1 wherein the animal has a higher risk of developing an infectious disease.

9. The method of claim 8 wherein the infectious disease is hepatitis, syphilis or leprosy.

10. The method of claim 1 wherein the substance containing NeuGc is autogenic or allogeneic cells, and wherein the cells contain NeuGc on their plasma membrane.

11. The method of claim 10 wherein the NeuGc-containing autogenic or allogeneic cells are produced by incubating cells with sialyltransferase using CMP-NeuGc as a substrate.

12. The method of claim 10 wherein the NeuGc-containing autogenic or allogeneic cells are produced by transfecting cells with the CMP-NeuAc hydroxylase cDNA.

13. The method of claim 10 wherein the NeuGc-containing autogenic or allogeneic cells are produced by adding a synthetic precursor of CMP-NeuGc to cells under cell culture conditions.

14. The method of claim 13 wherein the synthetic precursor of CMP-NeuGc is N-glycolyl mannosamine pentaacetate.

15. The method of claim 10 wherein the NeuGc-containing autogenic or allogeneic cells are produced by incorporating NeuGe-rich gangliosides into the plasma membrane of the cells.

16. The method of claim 10 wherein the cells are red blood cells or other cells from humans and birds.

17. The method of claim 10 wherein the substance is administered by intravenous injection.

18. The method of claim 1 wherein the substance containing NeuGc is NeuGc-containing tissue or cells from animals.

19. The method of claim 18 wherein the animal is any animal except humans and birds.

20. The method of claim 18 wherein tissue or cells from animals is brain tissue or red blood cells.

21. The method of claim 18 wherein the substance is administered by oral administration.

22. A method of treating cancer in a human by increasing the anti-NeuGc antibody level in the blood of the human comprising administrating to the human an effective amount of a composition comprising autogenic or allogeneic cells, wherein the cells contain NeuGc on their plasma membrane.

23. Red blood cells from humans or birds, wherein the cells contain NeuGc on their plasma membrane.

24. A vaccine composition for increasing anti-NeuGc antibody level in the blood of an animal comprising a substance containing NeuGc.

25. The vaccine composition of claim 24 wherein the substance containing NeuGc is autogenic or allogeneic cells, and wherein the cells contain NeuGc on their plasma membrane.

26. The vaccine composition of claim 24 wherein the substance containing NeuGc is NeuGc-containing tissue or cells from animals except humans and birds.
